(19) United States
(12) Patent Application Publication
(10) Pub. No.: US 2021/0074398 A1
(43) Pub. Date: Mar. 11, 2021

(54) EVALUATION OF PATIENT SAFETY EVENT REPORTS FROM FREE-TEXT DESCRIPTIONS

(71) Applicant: MedStar Health, Inc., Columbia, MD (US)

(72) Inventors: Allan Fong, Washington, DC (US); Katherine Adams, Washington, DC (US); Jessica Howe, Washington, DC (US); Raj Ratwani, Washington, DC (US)

(21) Appl. No.: 17/016,641

(22) Filed: Sep. 10, 2020

Related U.S. Application Data
(60) Provisional application No. 62/898,213, filed on Sep. 10, 2019.

(51) Int. Cl. G16H 15/00; G16H 10/60; G16H 40/20; G16H 50/20; G16H 50/70; G06N 20/00; G06F 16/28; G06F 40/30

(52) U.S. Cl. CPC G16H 15/00; G16H 10/60; G16H 40/20; G06F 40/30; G16H 50/70; G06N 20/00; G06F 16/285; G16H 50/20

(57) ABSTRACT

Systems and methods are provided for classifying a patent safety event report into one or more of a plurality of clinically relevant output classes. A patient safety event report is received from a health information technology (HIT) system. A plurality of features representing the semantic content of a free-text narrative associated with the patient safety event report is extracted. The patent safety event report is classified into one or more of a plurality of clinically relevant output classes from at least the plurality of features at a machine learning model.

### RELATED APPLICATIONS

This application claims priority from U.S. Provisional Application No. 62/898,213, filed 10 Sep. 2019 and entitled “EVALUATION OF PATIENT SAFETY EVENT REPORTS FROM FREE-TEXT DESCRIPTIONS,” the subject matter of which is incorporated herein by reference in its entirety.

### TECHNICAL FIELD

This invention relates to electronic medical records systems, and more particularly, to evaluation of patient safety event reports from free-text descriptions.

### BACKGROUND

Despite an increased focus on patient safety, medical errors remain a leading cause of death in the United States. While there is widespread adoption of patient safety reporting systems, making sense of the data provided by these systems is still very challenging in practice. A main challenge is the categorization of these reports and challenging taxonomies used to describe patient safety events. This leads to many reports categorized as miscellaneous (or other) that would need to either be manually recategorized or ignored.

Further, the widespread use of health information technology (HIT), including electronic health records (EHRs) has improved certain aspects of patient care, but has also resulted in unintended safety consequences. Many of these safety hazards are associated with the usability of the technology and these hazards can lead to patient harm or even death. Improving patient safety is a top priority for nearly every healthcare provider organization. Unsafe care leads to patient harm and unnecessary cost.

### SUMMARY

In accordance with an aspect of the invention, a method is provided. A patient safety event report is received from a health information technology (HIT) system. A plurality of features representing the semantic content of a free-text narrative associated with the patient safety event report is extracted. The patent safety event report is classified into one or more of a plurality of clinically relevant output classes from at least the plurality of features at a machine learning model.

In accordance with another aspect of the invention, a system includes a processor, an output device, and a non-transitory computer readable medium that stores machine executable instructions for classifying a patent safety event report into one or more of a plurality of clinically relevant output classes. The machine executable instructions include a network interface that receives a patient safety event report from a health information technology (HIT) system and a feature extractor that extracts a plurality of features representing the semantic content of a free-text narrative associated with the patient safety event report. A machine learning model assigns the patent safety event report to at least one of a plurality of clinically relevant output classes from at least the plurality of features. A user interface displays the selected clinically relevant output classes to a user at the output device.

In accordance with a further aspect of the invention, a method is provided. A patient safety event report is received from a health information technology (HIT) system. A plurality of features representing the semantic content of a free-text narrative associated with the patient safety event report are extracted. The patent safety event report is assigned into one of a plurality of clinically relevant output classes representing a tone of the report. The patent safety event report is provide to a supervisor for review if the assigned class is one of a predetermined subset of the plurality of clinically relevant output classes.

### BRIEF DESCRIPTION OF THE DRAWINGS

FIG. 1 illustrates a system for evaluating patient safety event reports in a health information technology system in accordance with an aspect of the present invention;

FIG. 2 illustrates a method for categorizing a patient safety event report into at least one clinically relevant output class in accordance with an aspect of the present invention;

FIG. 3 illustrates a method for categorizing a patient safety event report into one of a plurality of classes representing an event type associated with an event that is the subject of the patient safety event report in accordance with an aspect of the present invention;

FIG. 4 illustrates a method for categorizing a patient safety event report into one or more of a plurality of classes representing casual factors associated with an event that is the subject of the patient safety event report in accordance with an aspect of the present invention;

FIG. 5 illustrates a method for indexing patient safety event reports in accordance with an aspect of the present invention;

FIG. 6 illustrates a method for categorizing a patient safety event report into one of a plurality of classes representing a tone of the patient safety event report; and

FIG. 7 is a schematic block diagram illustrating an exemplary system of hardware components for implementing the systems and methods described herein.

### DETAILED DESCRIPTION

The focus of improving patient safety has led to the adopting of patient safety events at many healthcare systems across the United States. These reports, which are referred to generally as incident reports, adverse events, or near misses and referred to herein as “patient safety reports,” generally contain structured elements such as the event category (e.g. medication, fall) and severity level (e.g., near miss, reached patient, reached patient with harm), as well as an unstructured free-text narrative that provides a description of the event with possible contributing factors. These reports hold tremendous promise for identifying safety hazards and then later monitoring whether a safety risk has been mitigated. However, there are several challenges with patient safety event report systems, including unclear taxonomies, which affects how reports are categorized. Even with an agreed upon common format for top-level event taxonomy, it is up to individual facilities to customize their taxonomy, and even when the taxonomies are similar, there is no guarantee that people will understand or use them. As a result, a large number of reports may be designed as “miscellaneous” or “other” in place of a more appropriate event category. In fact, miscellaneous categories are consistently reported as among the top five most frequently selected categories.

In addition, the general categories used in patient safety event reports can mask underlying causes for events, such as difficulties with health information technology (HIT) systems. Many frontline clinicians do not properly categorize HIT related events in the HIT category, making it difficult to identify the reports that are actually HIT related. With provider organizations accumulating tens of thousands of these reports, it is increasingly difficult for patient safety analysts, who are often responsible for the integration, analysis, dissemination, and management of patient safety event reports, to read each report to determine whether it is related to HIT usability and safety. As a result, HIT and usability issues are generally underreported relative to other types of events. For example, recent research focused on medication-related patient safety events has found that a majority of these events can be attributed to computerized provider order entry.

Identifying the specific HIT usability and safety hazards associated with patient harm can be challenging given that HIT is now intertwined with the care delivery process and many hazards that may be associated with HIT may not be easily related back to the HIT system. For example, a physician may make an error and enter the incorrect medication dose when ordering through the EHR, because of a confusing display, and that error may be later caught by the nurse attempting to administer the medication. This near miss event is often documented as a medication error with no mention of the HIT system that may be associated with the safety event.

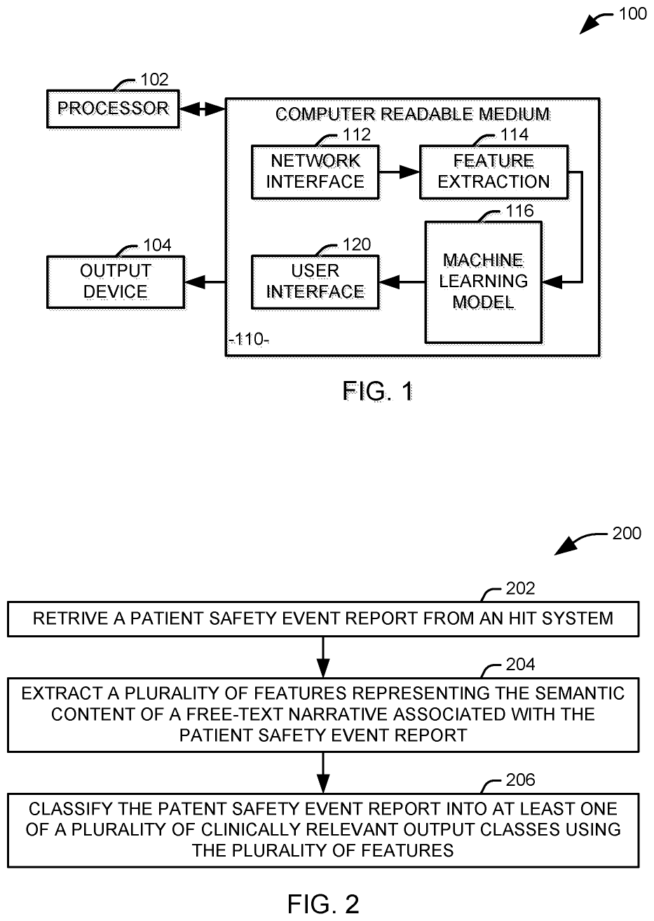

FIG. 1 illustrates a system 100 for evaluating patient safety event reports in a health information technology (HIT) system in accordance with an aspect of the present invention. Specifically, the system 100 can categorize each safety report into a clinically relevant category. It will be appreciated that a “clinical relevant category” can include any category relevant to clinical practice within a health care facility, and is explicitly intended to include categories relevant to a cause of the incident leading to the safety report, a tenor of the safety report (e.g., positive and result-oriented, negative or blame-shifting, or neutral), a type or class of the patient safety event, a type or class of medication associated with the incident leading to the safety report, and a degree of involvement of HIT systems in the incident leading to the safety report. It will further be appreciated that, in many implementations, a given safety report can be classified into multiple categories, with degrees of class membership. For example, where the classes represent specific causes of the incident, a given safety report can be classified into multiple categories to generate a profile of causative factors for the incident.

The system 100 includes a processor 102, an output device 104, and a non-transitory computer readable medium 110 storing computer readable instructions, executed by the processor 102. The executable instructions stored on the non-transitory computer readable medium 110 include a network interface 112 via which the system 100 communicates with other systems (not shown) via a network connection, for example, an Internet connection and/or a connection to an internal network. In the illustrated example, the other systems can include an electronic health records (EHR) system that stores medical information for the patient as well as terminals within a health care facility for accessing and displaying information produced by the system 100. It will be appreciated that the system 100 can be implemented as a virtual or cloud server, in which case the processor 102 and the non-transitory computer readable medium 110 may be shared by other applications.

Patient safety event reports can be extracted from the HIT system (not shown) via the network interface 112 and provided to a feature extractor 114 that extracts a plurality of features for use at a machine learning model 116. Patient safety event reports generally contain structured elements like general event types and free-text narratives written by the reporting medical professional. In accordance with an aspect of the present invention, at least some of the features used for classifying the safety report can be drawn from the free-text narrative field from the report. To this end, the feature extractor 114 can utilize one or more natural language processing algorithms for extracting data from unstructured text as well as appropriate templates and queries for extracting specific fields from semi-structured data sources. While the structured data fields (e.g., reported cause, reported location, etc.) can be relevant in classifying the patient safety event report, in the illustrated implementation, the system 100 focuses on the semantic content of the free-text narrative, and may even replace existing structured fields according to the evaluation of the free-text.

In one example, a bag-of-words approach is utilized. In the bag-of-words approach, each report is represented as a feature vector generated according to the frequency of terms within the report. The bag-of-words features can be weighted using the token frequency according to term frequency-inverse document frequency (tfidf), such that terms that occur relatively infrequently across reports are accorded more weight per occurrence than more common terms. In another example, a topic modeling approach is utilized, in which latent topics in the patient safety event report text can be identified to provide additional data for classification. For example, a patient safety event report classified as a “fall” by a frontline reporter could include latent “medication” and “provider fatigue” themes or topics in the free-text. Topic modeling is an unsupervised method to detect these topics, which can be used as additional information for classifying events. In one example, the feature extractor 114 can utilize latent Dirichlet allocation, which is a generative topic model that discovers topics in textual documents. Once an appropriate set of latent topics are identified during training of the system 100, the feature extractor 114 can transform each report into a topic representation formed from the latent topics expected to generate the words observed in the report.

In another example, a word embedding, such as Word2Vec, or a document embedding approach, such as Doc2Vec can be used. Document embedding is an unsupervised algorithm that learns word sequence features from variable-length documents or reports resulting in dense vector representations of documents. The interesting property of these vectors, as compared with sparse bag-of-words features, is that they can capture semantic and syntactic relatedness between the words and word sequences in a dense representation. Therefore, document embeddings could represent the dependencies and interactions between word sequences across documents. In this approach, the feature extractor 114 generates a document vector of the free-text of a patient safety event report that captures embedding representations averaged across occurring words and word sequences. It will be appreciated that these approaches are not exclusive, and that multiple approaches can be utilized in generating classification features.

The machine learning model 116 uses the extracted features to classify a novel patient safety event report, that is, an event report that was not presented in a training set for the model, into one or more of a plurality of clinically relevant classes. The machine learning model 116 can utilize one or more pattern recognition algorithms, implemented, for example, as classification and regression models, each of which analyze the extracted features or a subset of the extracted features to classify the patients into one of the plurality of clinically relevant classes. The selected class can be provided to a user at an associated display (not shown) or stored on the non-transitory computer readable medium 110, for example, in a record associated with the patient safety event report. Where multiple classification and regression models are used, the machine learning model 116 can include an arbitration element can be utilized to provide a coherent result from the various algorithms. Depending on the outputs of the various models, the arbitration element can simply select a class from a model having a highest confidence, select a plurality of classes from all models meeting a threshold confidence, or select a class via a voting process among the models. Alternatively, the arbitration element can itself be implemented as a classification model that receives the outputs of the other models as features and generates one or more output classes for the patient safety event report. Once the output class or classes have been assigned at the machine learning model 116, the selected class or classes can be displayed to the user at the output device 104 via a user interface 118.

The machine learning model 116, as well as any constituent models, can be trained on training data representing the various classes of interest. It will be appreciated that, where multiple models are used, a given model may not consider all of the plurality of the output classes associated with the machine learning model 116 as a whole. In one implementation, the machine learning model 116 can use a plurality of individual models that each generate a confidence for a single class, with the arbitration component selecting either the class associated with the model with the highest confidence or all classes associated with models producing a confidence value above a selected threshold value. The training process of a given model will vary with its implementation, but training generally involves a statistical aggregation of training data into one or more parameters associated with the output classes. Any of a variety of techniques can be utilized for the models, including support vector machines, regression models, self-organized maps, fuzzy logic systems, data fusion processes, boosting and bagging methods, rule-based systems, or artificial neural networks.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. An SVM classifier utilizes a user-specified kernel function to organize training data within a defined feature space. In the most basic implementation, the kernel function can be a radial basis function, although the systems and methods described herein can utilize any of a number of linear or non-linear kernel functions.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier.

A regression model applies a set of weights to various functions of the extracted features, most commonly linear functions, to provide a continuous result. In general, regression features can be categorical, represented, for example, as zero or one, or continuous. In a logistic regression, the output of the model represents the log odds that the source of the extracted features is a member of a given class. In a binary classification task, these log odds can be used directly as a confidence value for class membership or converted via the logistic function to a probability of class membership given the extracted features.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used.

In one implementation, the machine learning model 116 includes an ensemble model comprises a plurality of binary classifiers each trained to output the probability of a patient safety report belonging to the class represented by the binary classifier. Each binary classifier was implemented as a support vector machine classifier using a linear kernel function. To generate probabilities from the outputs of the linear support vector machines, a probability calibration, such as Platt's calibration, is performed with sigmoid functions. The one or more classes having the highest probability can be selected as the output class or classes for the machine learning model 116. Alternatively, every class having a probability value above a threshold value can be selected to provide the output classes.

In one implementation, the machine learning model 116 assigns a probability to the patient safety event report indicating the single category label that best matches the free-text narrative. Example event types can include events impacting the safety and health of patients that are related to medication or fluid, labs or specimens, falls, miscellaneous, blood banks, diagnosis or treatment, patient identification, documentation, or consent, surgeries or other procedures, skin or tissue, lines, tubes, or drains, safety or security, diagnostic imaging, professional conduct, equipment or medical devices, maternal care or childbirth, airway management, infection prevention, facilities, healthcare information technology, or injury due to restraints or seclusion. Each event type represents one output class. This implementation is of particular use in recategorizing events designated as miscellaneous, as the inventors have found that around ten percent of all patient safety reports are designated as miscellaneous, making it one of the three most used categories for the reports. This can also be used predictively to suggest a category to a user filling out a patient safety event report based on the semantic content of the free-text narrative.

In another implementation, probabilities are assigned to multiple categories to identify multiple best matches creating a profile for the report. It will be appreciated that some incidents do not fall cleanly into a single one of the categories presented above. For example, a patient fall caused by the side effects of an administered medication could easily be categorized as a “fall” or a “medication or fluid” event. The use of multiple classes allows for flexibility in assigning classes to the reports for more complex incidents. In practice, this implementation can be performed in conjunction with the single class implementation described above to provide a profile of the report based on the individual likelihoods of the report being associated with different event classes and a recommended categorization of the report with an aggregate probability score for the model. The machine learning model 116 can also be applied to determine a tenor or sentiment of the free-text report. Specifically, the free-text can be analyzed to classify the report as positive, negative, or neutral in sentiment, taking into consideration ambiguous words. Knowing the sentiment of a report provides insight into potential issues in the facility itself, such the state of workplace culture. In one implementation, the output is a score from −1, representing a negative sentiment or blame-oriented workplace culture, to 1, representing a positive sentiment or encouraging workplace culture, for the report as well as the parts of the free-text associated with the score.

The classes can also represent likely causal factors for a patient safety event associated with the report, such as distraction/interruptions, a noisy environment, communication breakdowns, and similar causes, as well as the parts of the free-text associated with the factors. It will be appreciated that the specific causal factors used for this purpose will vary with the implementation of the system and the environment in which it is employed. In one implementation, events that have been categorized as “Health IT” can be evaluated to determine if the event was usability related, that is, if the user interface of the health IT interface may have been a contributing factor in the event. The classes can also represent specific events instead of general event classes. In this implementation, the output can include likely specific events identified in the report, incorrect dosages, patient identification errors, “wrong side” errors, incorrect procedures, and similar events, as well the parts of the free-text associated with the specific events.

In still another implementation, the detected semantic features can include medication names, including variant spellings and brand names, identified within the free-text, and the report can be classified into classes representing specific drugs and drug classes. For example, a patient safety report that mentions providing the patient with “eloquiz”, could be mapped to any or all of the proper spelling of the brand name “Eliquis,” the medication name “apixaban,” and the general category of blood thinners. The frequency of specific medications and drug classes being mentioned across reports can be identified to determine the frequency with which medications are administered and the frequency with which various medications are involved in a patient safety event. Since the determined category can be associated with the report in the HIT database in which it is stored, this implementation also allows for a more robust search function, since misspellings, synonyms, and drugs from the same class can be identified.

A similar process can be applied to classify the report based on commonly misspelled or confused words as well as word tense differences within the free-text. Key words or variants on those key words can be extracted to provide a feature representation for each report. The report can then be indexed with these key words, allowing other similar reports to be identified to find temporal trends associated with safety events and hazards. This allows for a more intuitive search process for the patient safety reports and increases the number of relevant results returned, as minor spelling and tense differences are accounted for.

In a further implementation, the machine learning model 116 can determine whether or not a given incident is associated with a given event type. For example, incidents involving misunderstandings of healthcare information technology systems can often be categorized as a different type of error. For example, a physician misreading a HIT patient record might prescribe an incorrect dosage of a medication, leading to a “medication or fluid” event or a nurse misreading a patient's medical history in an HIT patient record might take insufficient precautions, leading to a “fall” event, but in each case, a miscomprehension of the information provided by the HIT system was a causal factor in the event. By failing to categorize these events as HIT events, opportunities to clarify the HIT interface and avoid future errors may be missed.

In view of the foregoing structural and functional features described above, methods in accordance with various aspects of the present invention will be better appreciated with reference to FIGS. 2-6. While, for purposes of simplicity of explanation, the methods of FIGS. 2-6 are shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a method in accordance with an aspect the present invention.

FIG. 2 illustrates a method 200 for categorizing a patient safety event report into at least one clinically relevant output class in accordance with an aspect of the present invention. At 202, a patient safety event report is retrieved from a health information technology (HIT) system. For example, the patient safety event report can be retrieved via an appropriate query of a HIT database or automatically received from the HIT database upon generation of the patient safety event report. At 204, a plurality of features representing the semantic content of a free-text narrative associated with the patient safety event report are extracted. The feature extraction performed on the free-text narrative can utilize any of a number of natural language processing techniques to extract features useful for classification, including, but not limited to, a term frequency-inverse document frequency weighted bag-of-words approach, topic modelling, word embedding, and document embedding techniques.

At 206, the patent safety event report is classified into at least one of a plurality of clinically relevant output classes from at least the plurality of features. Each output class can represent, for example, a general type of patient safety event, a specific patient safety event, a causal factor of a patient safety event, a type or class of drug involved in a patient safety event, a sentiment or tenor of the free-text narrative, or a word or topic associated with the patient safety event that may be useful for indexing purposes. It will be appreciated that the classification can be performed via a machine learning model trained on data representing the plurality of clinically relevant output classes. In one implementation, the machine learning model uses an ensemble of binary (i.e., single class) classifiers, each implemented as one of a support vector machine, a logistic regression model, or a random forest classifier and selects a class or classes according to confidence values produced by the various models.

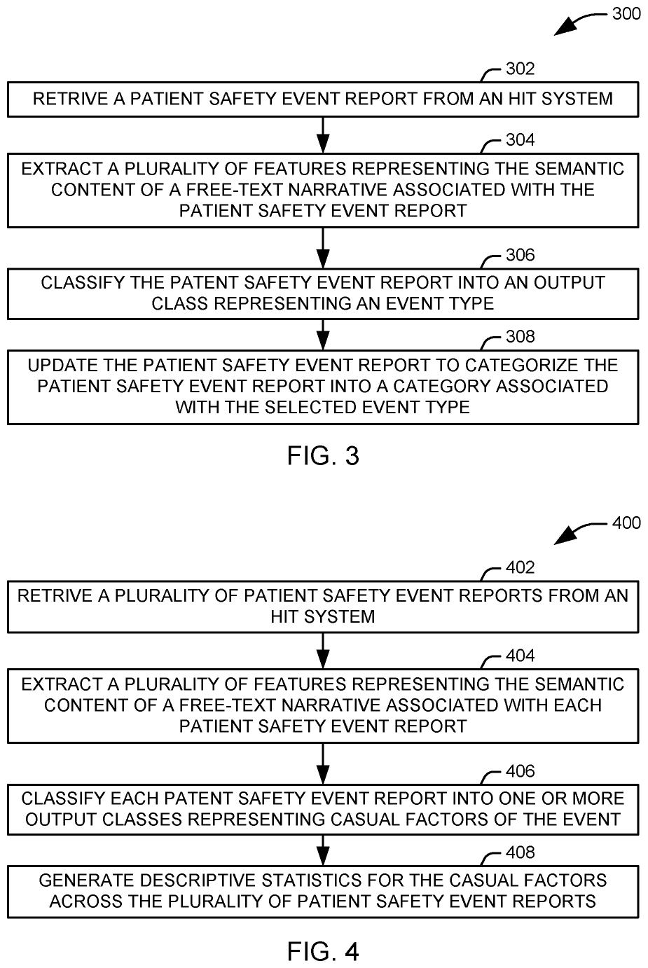

FIG. 3 illustrates a method 300 for categorizing a patient safety event report into one of a plurality of classes representing an event type associated with an event that is the subject of the patient safety event report in accordance with an aspect of the present invention. At 302, a patient safety event report is retrieved from a health information technology (HIT) system. At 304, a plurality of features representing the semantic content of a free-text narrative associated with the patient safety event report are extracted using natural language processing techniques which can include any of a term frequency-inverse document frequency weighted bag-of-words approach, topic modelling, word embedding, and document embedding techniques.

At 306, the patent safety event report is assigned to a class representing an event type. Example event types can include events impacting the safety and health of patients that are related to medication or fluid, labs or specimens, falls, miscellaneous, blood banks, diagnosis or treatment, patient identification, documentation, or consent, surgeries or other procedures, skin or tissue, lines, tubes, or drains, safety or security, diagnostic imaging, professional conduct, equipment or medical devices, maternal care or childbirth, airway management, infection prevention, facilities, healthcare information technology, or injury due to restraints or seclusion. At 308, the patient safety event report is updated to categorize the patient safety event report into a category associated with the selected event type.

FIG. 4 illustrates a method 400 for categorizing a patient safety event report into one or more of a plurality of classes representing casual factors associated with an event that is the subject of the patient safety event report in accordance with an aspect of the present invention. At 402, a plurality of patient safety event reports are retrieved from a health information technology (HIT) system. At 404, a plurality of features representing the semantic content of a free-text narrative associated with the patient safety event report are extracted from each patient safety event report using natural language processing techniques which can include any of a term frequency-inverse document frequency weighted bag-of-words approach, topic modelling, word embedding, and document embedding techniques.

At 406, each of the plurality of patent safety event reports is assigned to one or more classes representing causal factors of the event. Example casual factors can include distraction/interruptions, a noisy environment, communication breakdowns, and poor user interfaces in HIT systems. At 408, descriptive statistics representing the casual factors across the plurality of patient safety event reports are generated to represent the prevalence of various casual factors. When a particular casual factor is found to be prevalent at a given facility, processes for various procedures can be reevaluated and employees can be retrained to reduce the occurrence of the casual factors.

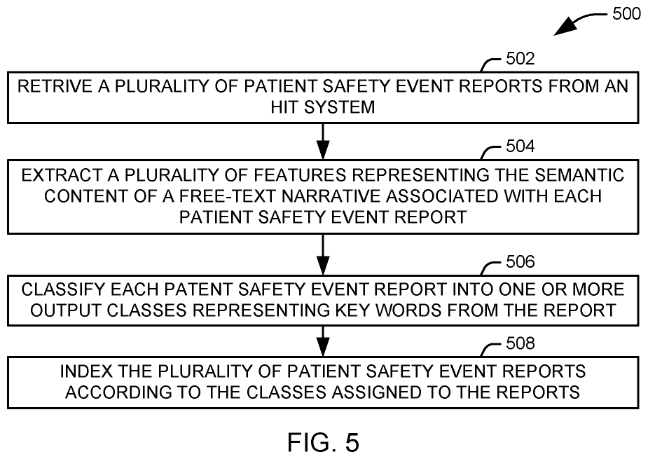

FIG. 5 illustrates a method 500 for indexing patient safety event reports in accordance with an aspect of the present invention. At 502, a plurality of patient safety event reports are retrieved from a health information technology (HIT) system. At 504, a plurality of features representing the semantic content of a free-text narrative associated with the patient safety event report are extracted from each patient safety event report using natural language processing techniques which can include any of a term frequency-inverse document frequency weighted bag-of-words approach, topic modelling, word embedding, and document embedding techniques.

At 506, each of the plurality of patent safety event reports is assigned to one or more classes representing key words within the patent safety event report. In addition to standard words used for indexing, the key words can include commonly misspelled or confused words as well as word tense differences to correctly index reports even when the free-text field may contain errors. Similarly, key words can include variants on common terms, such as names, brand names, and general categories for medications. At 508, the plurality of patient safety reports are indexed according to the assigned one or more classes.

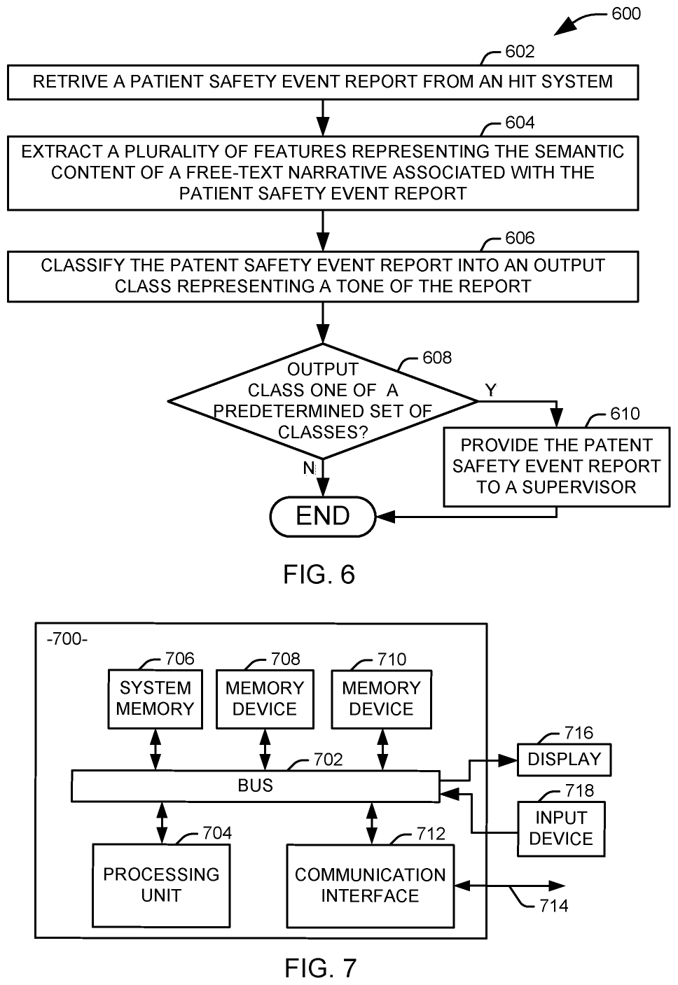

FIG. 6 illustrates a method 600 for categorizing a patient safety event report into one of a plurality of classes representing a tone of the patient safety event report. At 602, a patient safety event report is retrieved from a health information technology (HIT) system. At 604, a plurality of features representing the semantic content of a free-text narrative associated with the patient safety event report are extracted using natural language processing techniques which can include any of a term frequency-inverse document frequency weighted bag-of-words approach, topic modelling, word embedding, and document embedding techniques.

At 606, the patent safety event report is assigned to a class representing a tone of the patient safety event report. For example, a given report can be classified into a first class, representing a positive tone, a second class, representing a neutral tone, or a third class, representing a negative tone. It will be appreciated that more than three classes can be used to capture additional gradations or nuance in the tone of the report. At 608, it is determined if the patient safety event report belongs to a predetermined subset of the plurality of output classes. For example, the subset can represent classes associated with a negative tone. If not (N), the method terminates. If so (Y), the method advances to 610, where a supervisor is provided with the patient safety event report for review. By evaluating the tone of the reports, issues in the workplace environment and tensions between employees can be detected and addressed before they can negatively impact patient care.

FIG. 7 is a schematic block diagram illustrating an exemplary system 700 of hardware components capable of implementing examples of the systems and methods disclosed herein. The system 700 can include various systems and subsystems. The system 700 can be a personal computer, a laptop computer, a workstation, a computer system, an appliance, an application-specific integrated circuit (ASIC), a server, a server BladeCenter, a server farm, etc.

The system 700 can include a system bus 702, a processing unit 704, a system memory 706, memory devices 708 and 710, a communication interface 712 (e.g., a network interface), a communication link 714, a display 716 (e.g., a video screen), and an input device 718 (e.g., a keyboard, touch screen, and/or a mouse). The system bus 702 can be in communication with the processing unit 704 and the system memory 706. The additional memory devices 708 and 710, such as a hard disk drive, server, standalone database, or other non-volatile memory, can also be in communication with the system bus 702. The system bus 702 interconnects the processing unit 704, the memory devices 706-710, the communication interface 712, the display 716, and the input device 718. In some examples, the system bus 702 also interconnects an additional port (not shown), such as a universal serial bus (USB) port.

The processing unit 704 can be a computing device and can include an application-specific integrated circuit (ASIC). The processing unit 704 executes a set of instructions to implement the operations of examples disclosed herein. The processing unit can include a processing core.

The additional memory devices 706, 708, and 710 can store data, programs, instructions, database queries in text or compiled form, and any other information that may be needed to operate a computer. The memories 706, 708 and 710 can be implemented as computer-readable media (integrated or removable), such as a memory card, disk drive, compact disk (CD), or server accessible over a network. In certain examples, the memories 706, 708 and 710 can comprise text, images, video, and/or audio, portions of which can be available in formats comprehensible to human beings. Additionally or alternatively, the system 700 can access an external data source or query source through the communication interface 712, which can communicate with the system bus 702 and the communication link 714.

In operation, the system 700 can be used to implement one or more parts of a system for categorizing patient safety event reports in accordance with the present invention, in particular, the feature extractor 114 and the predictive model 116. Computer executable logic for implementing the report categorization system resides on one or more of the system memory 706, and the memory devices 708 and 710 in accordance with certain examples. The processing unit 704 executes one or more computer executable instructions originating from the system memory 706 and the memory devices 708 and 710. The term “computer readable medium” as used herein refers to a medium that participates in providing instructions to the processing unit 704 for execution. This medium may be distributed across multiple discrete assemblies all operatively connected to a common processor or set of related processors.

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, and volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The terms “computer readable medium” and “machine readable medium” includes, but is not limited to portable or fixed storage devices, optical storage devices, wireless channels, and/or various other storage mediums capable of storing that contain or carry instruction(s) and/or data. It will be appreciated that a “computer readable medium” or “machine readable medium” can include multiple media each operatively connected to a processing unit.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. A method comprising:
receiving a patient safety event report from a health information technology (HIT) system;
extracting a plurality of features representing the semantic content of a free-text narrative associated with the patient safety event report; and
classifying the patent safety event report into at least one of a plurality of clinically relevant output classes from at least the plurality of features at a machine learning model.

2. The method of claim 1, wherein the plurality of clinically relevant output classes each represent an event type associated with an event that is the subject of the patient safety event report.

3. The method of claim 1, wherein the patient safety event report is a first patient safety event report of a plurality of patient safety event reports, the method further comprising selecting a set of the patient safety event reports from the plurality of patient safety event reports that have been categorized in a particular event class, and selecting the first patient safety event report from the set of the patient safety event reports.

4. The method of claim 1, wherein the plurality of clinically relevant output classes each represent casual factors associated with an event that is the subject of the patient safety event report.

5. The method of claim 1, wherein the plurality of clinically relevant output classes each represent one of a name of a medication, a brand name of a medication, and a category of medication.

6. The method of claim 1, wherein the plurality of clinically relevant output classes each represent a tenor of the patient safety report.

7. The method of claim 1, wherein the plurality of clinically relevant output classes each represent a key word used in indexing the plurality of patient safety event reports.

8. The method of claim 1, wherein the plurality of clinically relevant output classes include a first class indicating that the event that is the subject of the patient safety event report is associated with a given event type and a second class indicating that the event that is the subject of the patient safety event report is not associated with a given event type.

9. The method of claim 1, wherein classifying the patent safety event report into at least one of a plurality of clinically relevant output classes comprises providing the plurality of features to each of a plurality of binary classifiers, each of the plurality of binary classifiers representing one of the plurality of classifiers, to provide a probability value representing each class and selecting the at least one of the plurality of clinically relevant output classes as a set of classes having the highest probabilities.

10. The method of claim 1, wherein classifying the patent safety event report into at least one of the plurality of clinically relevant output classes comprises providing the plurality of features to each of a plurality of binary classifiers, each of the plurality of binary classifiers representing one of the plurality of classifiers, to provide a probability value representing each class and selecting each class having a probability value exceeding a threshold value as the at least one of the plurality of clinically relevant output classes.

11. The method of claim 1, wherein extracting a plurality of features representing the semantic content of a free-text narrative associated with the patient safety event report comprises extracting the plurality of features via one of word embedding and document embedding.

12. The method of claim 1, wherein extracting a plurality of features representing the semantic content of a free-text narrative associated with the patient safety event report comprises extracting the plurality of features via latent Dirichlet allocation.

13. The method of claim 1, wherein extracting a plurality of features representing the semantic content of a free-text narrative associated with the patient safety event report comprises extracting the plurality of features via a bag-of-words approach.

14. A system comprising:
a processor;
an output device; and
a non-transitory computer readable medium storing machine executable instructions for classifying a patent safety event report into at least one of a plurality of clinically relevant output classes, the machine executable instructions comprising:
a network interface that receives a patient safety event report from a health information technology (HIT) system;
a feature extractor that extracts a plurality of features representing the semantic content of a free-text narrative associated with the patient safety event report;
a machine learning model that assigns the patent safety event report to at least one of a plurality of clinically relevant output classes from at least the plurality of features; and
a user interface that displays the at least one of the plurality of clinically relevant output classes to a user at the output device.

15. The system of claim 14, wherein the machine learning model comprises a plurality of classifiers, each of the plurality of classifiers being trained to produce a probability that the patient safety event report belongs to an associated one of the plurality of output classes.

16. The system of claim 15, wherein the machine learning model assigns the patient safety event report into each class for which the probability that the patient safety event report belongs to that class exceeds a threshold value.

17. The system of claim 14, wherein the plurality of clinically relevant output classes each represent an event type associated with an event that is the subject of the patient safety event report.

18. The system of claim 14, wherein a feature extractor that extracts a plurality of features using one of a topic modelling approach, a word embedding approach, a document embedding approach, and a bag-of-words approach.

19. The system of claim 14, wherein the plurality of clinically relevant output classes each represent a tenor of the patient safety event report, with a first class representing a positive tenor, a second class representing a neutral tenor, and a third class representing a negative tenor.

20. A method comprising:
receiving a patient safety event report from a health information technology (HIT) system;
extracting a plurality of features representing the semantic content of a free-text narrative associated with the patient safety event report;
assigning the patent safety event report into one of a plurality of clinically relevant output classes representing a tone of the report; and
providing the patent safety event report to a supervisor for review if the assigned class is one of a predetermined subset of the plurality of clinically relevant output classes.
